(19) United States
(12) Patent Application Publication
(10) Pub. No.: US 2012/0046583 A1
(43) Pub. Date: Feb. 23, 2012

(54) PORTABLE OR WEARABLE FRACTURE TREATMENT DEVICE

(76) Inventor: Shinichi NAKAMITSU, Kitakyushu-city (JP)

(21) Appl. No.: 13/206,108

(22) Filed: Aug. 9, 2011

(30) Foreign Application Priority Data
Aug. 20, 2010 (JP) 2010-184899

(51) Int. Cl. A61F 5/40; A61F 5/00

(52) U.S. Cl. 602/4; 602/20

(57) ABSTRACT

A portable or wearable fracture treatment device includes a splint unit that is attached to an affected part of a forearm of a patient and a part surrounding the affected part so that the affected part is supported along a longitudinal direction of the forearm, an upper arm attachment unit that attaches the splint unit to an upper arm of the patient, a fixing unit that fixes a part of the patient that is between the affected part of the forearm and an end of a hand of the patient, and a traction unit that is supported by the splint unit. The traction unit pulls the affected part of the forearm of the patient in a direction from the affected part of the forearm toward the hand of the patient by pulling the fixing unit in the direction from the affected part of the forearm toward the hand of the patient.

### BACKGROUND OF THE INVENTION

1. Field of the Invention

The present invention relates to a fracture treatment device used for treating a forearm fracture.

2. Description of the Related Art

Fracturing of a wrist joint or the surrounding part often occurs when a person falls and lands on his/her hand. Due to the aging population, the number of fragility fractures such as distal radius fractures has been increasing rapidly in recent years. Such a fracture is usually treated by using a cast. To perform a conservative treatment using a cast, it is necessary to restore bone fragments to their substantially original locations with a single manipulative reduction. If the reduction is not performed under anesthesia, a patient suffers from pain. Even if the reduction is successful and the fractured part is fixed by using a cast, the fracture may subsequently recur inside the cast. Although a cast is useful for effective nonsurgical treatment, frequent use of reduction using a cast is avoided due to the problems of recurrence and anesthesia.

With surgical treatment, it is possible to directly move and anatomically restore bones to their substantially original locations. However, with surgical treatment, use of an invasive procedure into a soft tissue such as a muscle is unavoidable, and a blood vessel, a tendon, or a nerve may be damaged. Surgical treatment involves a risk due to anesthesia or the like, and a foreign body such as one composed of a metal will remain in the body. It is preferable that surgical treatment be avoided for elderly patients, who are physically weak. Surgical treatment causes a patient psychological and physical stress, and is expensive. As described above, both cast treatment and surgical treatment have many problems.

Continuous traction treatment in a recumbent or supine position is used for treating bone fractures in children, in particular, for treating a fracture of an elbow joint or the surrounding part or a femoral fracture, which is difficult to treat. For example, FIG. 10 illustrates a known fracture treatment device used for treating a forearm fracture. The fracture treatment device includes a brace 61 that is made of a net and is attached to a finger connected to the affected forearm, and the brace 61 is pulled by a weight 63 through a rope or string 62. In the example of FIG. 10, pulley 64 is used as means for pulling the brace 61 substantially horizontally. A good result can be obtained with the traction treatment by adjusting the reduction direction and the traction force. A patient only experiences a little pain in the fractured part in traction treatment, because the positions of the patient's limbs are not moved during the treatment. Therefore, traction treatment has advantages over surgical treatment in that a patient does not feel pain or anxiety, the risk is small, and the treatment can be administered at low cost. Moreover, traction treatment has advantages over cast treatment in that the affected part can be observed by the naked eye, which is difficult in cast treatment in which the affected part is covered with a cast; and a side effect due to a cast can be avoided.

On the other hand, traction treatment has disadvantages in that it is necessary to place a large device and a weight near a bed or a bed rail, and it is necessary for a patient to remain in a recumbent or supine position for a long time. In particular, when traction treatment is used for an elderly patient, the patient may suffer from muscular atrophy, articular contracture, or dementia due to lying in bed for a long time.

### SUMMARY OF THE INVENTION

As described above, continuous traction treatment in a recumbent or supine position has advantages in that a patient suffers from only a little pain in the fractured part and anesthesia is not necessary. However, existing fracture treatment devices used for continuous traction treatment, which use a weight to generate a traction force, have the following problems. First, such a fracture treatment device needs to be placed at a bedside because the device is heavy due to the weight. Second, a patient's lifestyle is restricted because the patient has to be in a recumbent or supine position for a long time. In particular, the patient may feel considerable mental distress regarding using a bedpan in bed. Third, because a weight is used to generate a traction force, the traction direction and the magnitude of the traction force cannot be finely adjusted.

An object of the present invention, which has been achieved in view of the problems of the prior art described above, is to provide a portable or wearable fracture treatment device with which it is possible to perform continuous traction treatment of a forearm fracture while allowing a patient to have a comfortable daily life without requiring the patient to remain in a recumbent or supine position. Another object of the present invention is to provide a portable or wearable fracture treatment device with which it is possible to finely adjust the traction direction and the magnitude of traction force.

According to the present invention, a portable or wearable fracture treatment device used for treating a forearm fracture includes a splint unit that is attached to an affected part of a forearm of a patient and a part of the patient surrounding the affected part so that the affected part is supported along a longitudinal direction of the forearm (i.e., such that the affected part is supported substantially parallel to the longitudinal direction of the forearm), an upper arm attachment unit that attaches the splint unit to an upper arm of the patient so that the splint unit is supported by the upper arm of the patient, a fixing unit that fixes a part (for example, a finger) of the patient that is between the affected part of the forearm and an end of a hand of the patient, and a traction unit that is supported by the splint unit. The traction unit pulls the affected part of the forearm of the patient in a direction from the affected part of the forearm of the patient toward the hand of the patient by pulling the fixing unit in the direction from the affected part of the forearm of the patient toward the hand of the patient, in a state in which the forearm of the patient is flexed substantially horizontally with respect to an upper arm of the patient that extends substantially vertically (i.e., in a state in which the forearm and the upper arm of the patient are substantially perpendicular to each other around the elbow of the patient).

In the present specification, the term “affected part” refers to “fractured part”. The phrase “a part that is more distal than an affected part” refers to a part of the patient that is located farther from the heart (center of the body) than the affected part is. If the affected part is a forearm, the distal part may be a palm, a finger, or the like. The phrase “a part that is more proximal than an affected part” refers to a part of the patient that is nearer to the heart than the affected part is. If the affected part is a forearm, the proximal part may be an upper arm or the like. In the present specification, a part that is more distal than an affected part of a forearm has the same meaning as a part of the patient that is between the affected part and the end of a hand of the patient. In the present specification, “hand” includes “finger”. In the present specification, it is preferable that the traction unit be fixed to the splint unit (base unit) that is attached to an affected part of a forearm. It is preferable that the splint unit (base unit) have the function of a splint (a thin plate or a part of a cast, which is made of aluminum or plastic and is used for treatment by being attached to an arm or a leg along the longitudinal direction of the arm or the leg).

According to the present invention, a portable or wearable fracture treatment device used for treating a forearm fracture may include a distal base unit that is disposed on a part of a patient between an affected part of a forearm and an end of a hand of the patient, a proximal base unit that is disposed at a part of the patient between the affected part of the forearm and an upper arm of the patient, a distal attachment unit that attaches the distal base unit to the part of the patient between the affected part of the forearm and the end of the hand of the patient, a proximal attachment unit that attaches the proximal base unit to the part of the patient between the affected part of the forearm and the upper arm of the patient, and a traction unit. The traction unit has one end fixed to the proximal base unit and the other end fixed to the distal base unit. The traction unit pulls the affected part of the forearm of the patient in a direction from the affected part toward the hand of the patient with respect to the part of the patient between the affected part of the forearm and the upper arm of the patient by applying a predetermined force to the proximal base unit and the distal base unit so as to increase the distance therebetween.

The distal base unit, which is disposed at a position more distal than the fractured part, and the proximal base unit, which is disposed at a position more proximal than the fractured part and which serves as a counter traction unit, may be disposed on a single splint so as to face each other with the fractured part therebetween and thereby exert a reduction effect on the fractured part.

It is preferable that the portable or wearable fracture treatment device according to the present invention further include an attachment unit that attaches the splint unit to a trunk or a shoulder of the patient.

It is preferable that, in the portable or wearable fracture treatment device according to the present invention, the attachment unit be a belt that is worn around the trunk of the patient, the belt having a front side to which the splint unit is attachable, the front side being opposite to a side of the belt that faces the trunk of the patient.

It is preferable that, in the portable or wearable fracture treatment device according to the present invention, the attachment unit be an orthopedic appliance that is slung over a shoulder of the patient, the appliance having a front side to which the splint unit is attachable, the front side being opposite to a side of the appliance that faces the trunk of the patient.

It is preferable that, in the portable or wearable fracture treatment device according to the present invention, the traction unit include a mechanism for adjusting a traction force applied to the affected part, the mechanism adjusting a length of a wire or string whose end is fixed to the fixing unit.

It is preferable that, in the portable or wearable fracture treatment device according to the present invention, the traction unit include a motor that generates a force with which the fixing unit is pulled.

With the present invention, because the splint unit for supporting the traction unit and the upper arm attachment unit for attaching (connecting) the splint unit to the upper arm are provided, traction can be continuously performed without requiring the patient to remain in a certain position. As a result, the present invention has an advantage over existing traction treatment devices in that it is not necessary to require the patient to continue to lie in bed. That is, with the present invention, a patient can receive traction treatment of a fracture while leading a normal daily life the same as that before suffering from a fracture. Therefore, with the present invention, when treating a fracture, a patient is not required to lie in bed as in existing traction treatment, so that decrease in muscular strength or impairment of visceral function are prevented. Moreover, progress of dementia due to lying in bed for a long time, which is an inherent problem in treating fractures of elderly patients, can be prevented.

With the present invention, when the splint unit or a forearm (the forearm to which the splint unit is attached) is supported by a trunk or a shoulder of the patient by using a belt that is worn around the trunk or an orthopedic appliance that is slung over the shoulder, the patient can easily carry the fracture treatment device according to the present invention.

With the present invention, the traction unit, which is carried or attached to a patient by using the splint unit, may include a mechanism (such as a winch) that adjusts the length of the wire or string used for adjusting a traction force applied to the affected part or a motor that generates and adjusts a force with which the fixing unit is pulled. In this case, because a weight is not used as in existing fracture treatment devices, the size and weight of the entire device can be reduced, and the traction direction and the magnitude of traction force can be finely and accurately set and adjusted. In particular, in the case where a weight is not used as a power source of the traction unit, when a patient performs traction treatment by attaching the present device to his/her upper limb by him/herself, the traction force does not change even if the direction of the present device is changed, whereby an excessive force is not applied to the affected part and negative influence on the treatment is reduced.

### BRIEF DESCRIPTION OF THE DRAWINGS

FIG. 1 illustrates a portable or wearable fracture treatment device according to a first embodiment of the present invention as seen from above a patient.

FIG. 2 is a schematic view of the device of FIG. 1 as seen from the front of the patient.

FIG. 3 is a schematic view illustrating the patient wearing the device according to the first embodiment.

FIGS. 4A and 4B illustrate a first modification of the first embodiment, which uses a plurality of winches in a traction unit.

FIGS. 5A and 5B illustrate a second modification of the first embodiment, which uses, instead of a pulley, a mechanism using a wire or the like that extends through a substantially U-shaped tube having an inner surface made of a low-friction plastic.

FIGS. 6A to 6C illustrate a third modification of the first embodiment, which includes a clutch mechanism that is disposed in a transmitting unit between a traction unit and an affected part of the patient and that serves as a safety device for preventing an excessive traction force from acting on an affected part.

FIGS. 7A and 7B illustrate a fourth modification of the first embodiment, which uses a motor as a component of a traction unit.

FIGS. 8A and 8B illustrate a fifth modification of the first embodiment, with which it is possible to pull a wrist in a state in which the wrist is extended substantially parallel to the longitudinal direction of a forearm and to pull the wrist in a state in which the wrist is flexed with respect to the longitudinal direction of the forearm.

FIGS. 9A and 9B illustrate a portable or wearable fracture treatment device according to a second embodiment of the present invention.

FIG. 10 illustrates a fracture treatment device of prior art.

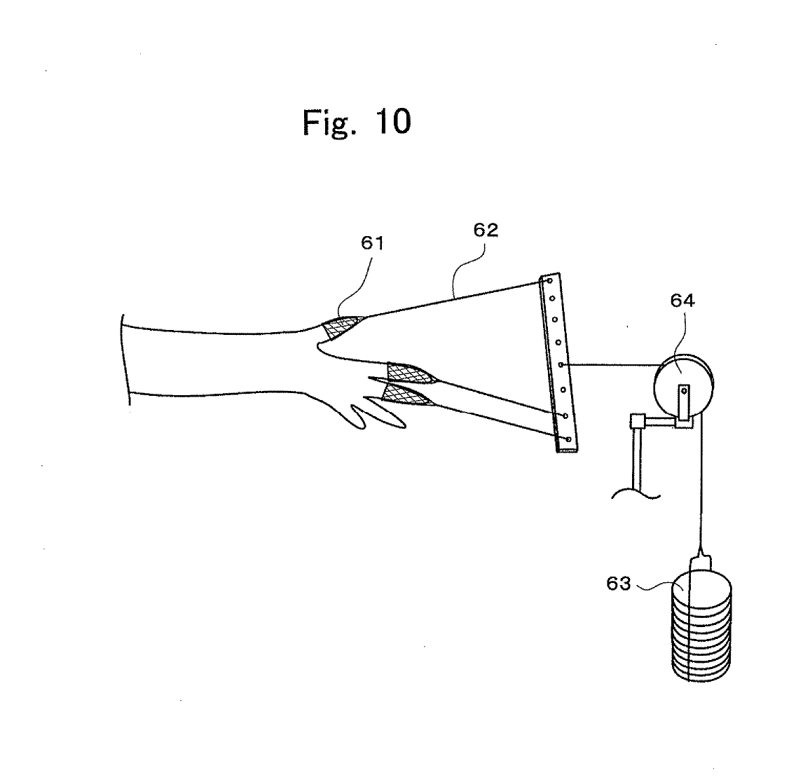

### DESCRIPTION OF THE PREFERRED EMBODIMENTS

A first embodiment of the present invention, which is the best mode for carrying out the present invention, will be described below.

### First Embodiment

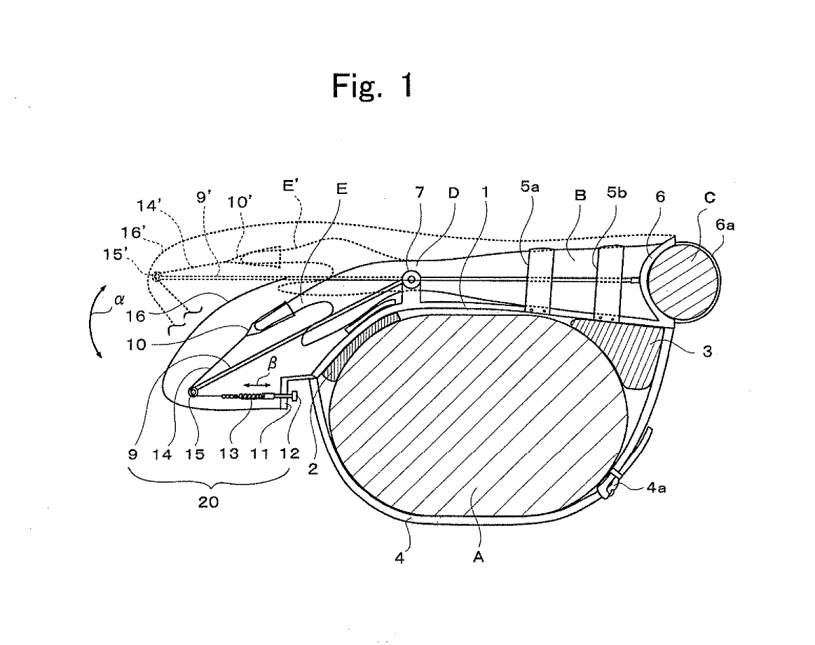

FIG. 1 illustrates a portable or wearable fracture treatment device according to the first embodiment of the present invention as seen from above a patient. FIG. 1 illustrates a trunk A of the patient, a forearm B of the patient, an upper arm C of the patient, a wrist D of the patient, and a finger E of the patient.

FIG. 1 illustrates a splint unit 1 (base unit), pads 2 and 3, a belt 4, and a buckle 4a of the belt 4. The splint unit 1 is made of a metal or a plastic. The splint unit 1 has a flat shape or a shape having a substantially U-shaped cross-section perpendicular to the longitudinal direction thereof (i.e., a shape that is curved so as to follow the surface of the forearm B of the patient). The pads 2 and 3 are made of a fabric or an elastic material such as a rubber and are fixed to the back surface of the splint unit 1 (which faces the trunk of the patient) with an adhesive or a hook-and-loop fastener (which is, for example, a hook-and-loop fastener marketed with the trademark “Velcro”). (The pads 2 and 3 are disposed on a left front part and on a right front part of the trunk A of the patient, respectively.) The belt 4 is fixed to both ends of the splint unit 1, and is worn around the outer peripheral surface of the trunk A of the patient. In the first embodiment, the splint unit 1 is fixed or attached to the trunk A of the patient over his/her clothes by using the belt 4. (In the first embodiment, instead of using the belt 4, the splint unit 1 may be fixed to the trunk A of the patient by sewing the splint unit 1 and the pads 2 and 3, which are fixed to the splint unit 1, onto the clothes of the patient.)

FIG. 1 illustrates belts 5a and 5b, both ends of which are fixed to the splint unit 1 and which are supported by being worn around the outer peripheral surface of the forearm B of the patient. (FIG. 1 does not illustrate buckles of the belts 5a and 5b.) The splint unit 1 is fixed or attached to the forearm B of the patient with the two belts 5a and 5b. FIG. 1 illustrates an upper arm attachment unit 6 that is made to contact an inner surface of the upper arm C of the patient (that faces the forearm B of the patient). The upper arm attachment unit 6 is attached to the upper arm C of the patient by using a belt 6a. The upper arm attachment unit 6 has a horizontal cross-section that is substantially semicircular so that the upper arm attachment unit 6 can easily contact the upper arm C of the upper arm C. In the first embodiment, the upper arm attachment unit 6 is integrally formed with the splint unit 1 at the right end of the splint unit 1 in FIG. 1. Although the upper arm attachment unit 6 is integrally formed with the splint unit 1 in FIG. 1, the upper arm attachment unit 6 and the splint unit 1 may be formed separately. The upper arm attachment unit 6 may include a mechanism for adjusting the size and angle thereof in accordance with the body size and preference of a user.

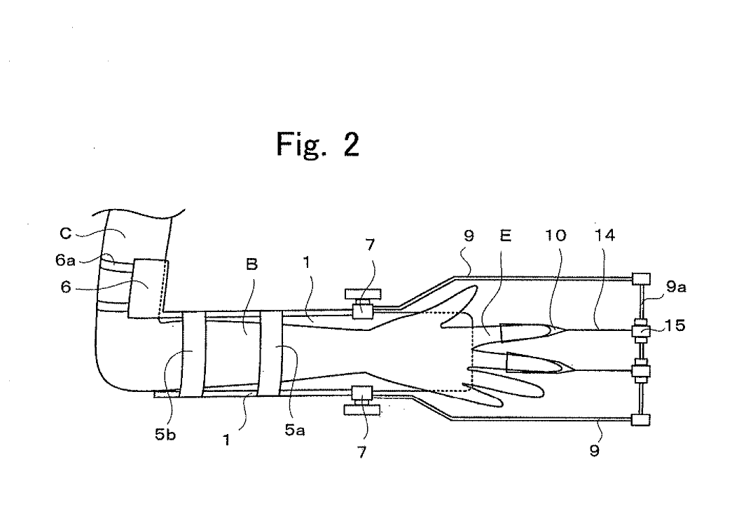

FIG. 1 illustrates a frame supporting unit 7 and a frame 9. The frame supporting unit 7 protrudes from a part of the splint unit 1 that faces the wrist D of the patient toward the wrist D of the patient. The frame 9 extends from the frame supporting unit 7 toward the finger E of the patient. The frame 9 is illustrated as a rod in FIG. 1. However, as illustrated in FIG. 2, the frame 9 actually includes two rods that extend substantially parallel to each other respectively above and below the hand of the patient. As illustrated in FIG. 2, the frame 9 includes a shaft 9a that extends vertically and connects the right ends of the two rods, and a pulley 15 that is attached to the shaft 9a. In the first embodiment, the frame 9 is connected to the frame supporting unit 7 so that the frame 9 can be rotated (the angle thereof can be changed) around the frame supporting unit 7 in directions in which the frame 9 moves toward or away from the trunk A of the patient of FIG. 1 (directions indicted by arrow α of FIG. 1) (see also broken lines denoted by numerals 9′, 10′, 14′, 15′, 16′, and E′ in FIG. 1).

FIG. 1 illustrates a brace 10, a hook-shaped protrusion 11, a knob screw 12, an elastic body 13, a wire or string 14, and the pulley 15. The brace 10 is fixed to the tip of the finger E of the patient, which is more distal than the fractured part (for example, the wrist of the patient). The brace 10 is a finger trap that has a basket-like shape formed by weaving bamboo or metal strings. When the brace 10 (the finger trap) is pulled, the inside diameter of the basket-like shape decreases, whereby the brace 10 fastens the tip of the finger E to be pulled. The hook-shaped protrusion 11 is fixed to the left end of the splint unit 1 in FIG. 1. The knob screw 12 is inserted (screwed) into a screw hole formed at substantially the center of the hook-shaped protrusion 11. One end of the elastic body 13 (which has markings) is fixed to an end of the knob screw 12. The wire or string 14 connects the other end of the elastic body 13 to an end of the brace 10. The pulley 15 is attached to the shaft 9a (see FIG. 2) of the frame 9 so as to change the direction in which the wire or string 14 is pulled. The length of the wire or string 14 from the brace 10 to the elastic body 13 can be adjusted by adjusting the depth to which the knob screw 12 is screwed into the screw hole in the hook-shaped protrusion 11, whereby the traction force applied to the brace 10 through the wire or string 14 can be adjusted. The elastic body 13, which serves to prevent a sudden change in the traction force, includes, for example, a spring or a rubber. (Instead of a rubber or a spring, the elastic body 13 may include a gas cylinder or the like). In the present invention, the knob screw 12 may be included in a winch mechanism that winds the wire or string 14 around a cylindrical body when the knob screw 12 is rotated, and the traction force of the wire or string 14 may be adjusted by using the winch mechanism.

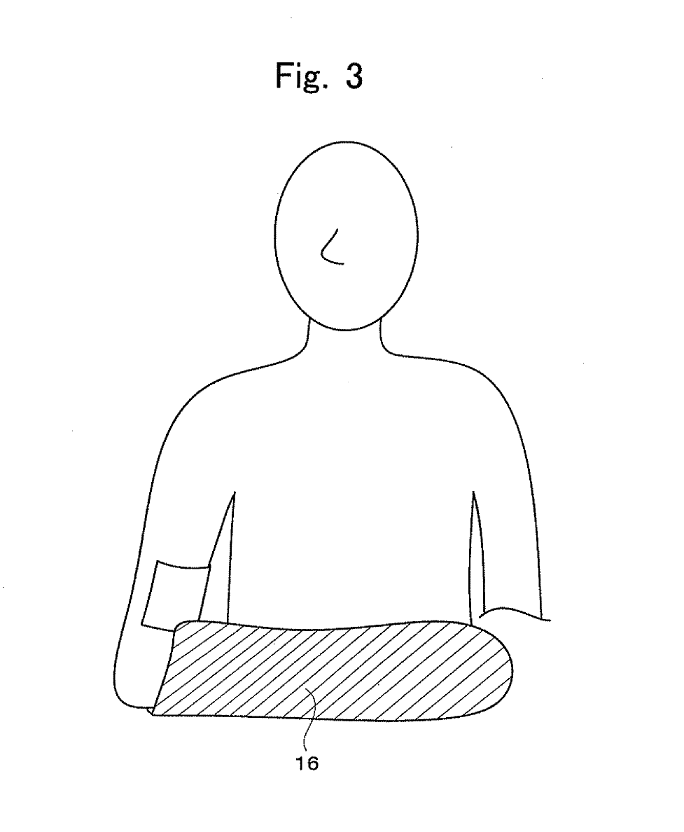

Referring to FIG. 1, in the first embodiment, the frame 9, the hook-shaped protrusion 11, the knob screw 12, the elastic body 13, the wire or string 14, and the pulley 15 constitute a traction unit 20 for pulling the affected part. FIG. 1 illustrates a cover 16 that is made of a fabric or a plastic sheet and that is attached to outer peripheral edges of the splint unit 1 and the hook-shaped protrusion 11. The cover 16 shields the traction unit 20 and the forearm of the patient so that they cannot be seen from the outside, and protects the forearm from direct contact with an external object (see FIG. 3).

As described above, the frame 9 is connected to the frame supporting unit 7 so that the frame 9 can be rotated (the angle thereof can be changed) around the frame supporting unit 7 in directions in which the frame 9 moves toward or away from the trunk A of the patient of FIG. 1 (i.e. in directions indicted by arrow α of FIG. 1) (see also broken lines denoted by numerals 9′, 10′, 14′, 15′, 16′, and B′ in FIG. 1). Therefore, in the first embodiment, as illustrated in FIG. 1, the direction in which the finger E is pulled (the direction in which the traction force is applied to the affected part) can be adjusted by rotating the frame 9 around the frame supporting unit 7 and changing the angle of the frame 9 with respect to the longitudinal direction of the forearm B. That is, whether to pull the wrist D in a state in which the wrist D is extended substantially parallel to the longitudinal direction of the forearm or to pull the wrist D in a state in which the wrist D is flexed with respect to the forearm can be selected.

FIG. 2 is a schematic view of the fracture treatment device according to the first embodiment illustrated in FIG. 1 as seen from the front of the patient. Because FIG. 2 is a schematic view, some parts are not strictly the same as those of FIG. 1. For example, FIG. 2 does not illustrate the cover 16 and the knob screw 12, which are illustrated in FIG. 1. FIG. 3 is a schematic view illustrating how the present embodiment is used, as seen from the front of the patient. In FIG. 3, the cover 16 almost entirely covers the splint unit 1, the traction unit 20, and the forearm of the patient, which are illustrated in FIG. 1. However, the first embodiment is not limited thereto. The cover 16 may cover only part of the splint unit 1, the traction unit 20, and the forearm of the patient.

Next, the operation and the method of using the first embodiment will be described. FIGS. 1 and 2 illustrate a case where the brace 10, which is attached to the tip of finger, is pulled. In this case, first, the splint unit 1 is made to contact the forearm B of the patient, and the belts 5a and 5b, which are fixed to the splint unit 1, are worn around the outer peripheral surface of the forearm B and then tightened, thereby the splint unit 1 is fixed or attached to the forearm B.

Next, the upper arm attachment unit 6, which is formed at the right end of the splint unit 1 in FIG. 1, is made to contact the upper arm C and is attached to the upper arm C with the belt 6a, and thereby the splint unit 1 is connected to the upper arm C. Thus, the splint unit 1 is supported by the forearm B and the upper arm C.

Next, the brace 10, to which end the wire or string 14 has been fixed beforehand, is attached to the tip of the finger E of the patient. Subsequently, the magnitude of the traction force transferred through the wire or string 14 to the finger E (traction force applied to the affected part) is adjusted by rotating the knob screw 12 and moving the wire or string 14 in the direction of arrow β in FIG. 1.

Next, the splint unit 1, which is fixed or attached to the forearm B of the patient, is fixed or attached to the trunk A of the patient over the pads 2 and 3 and clothes (not shown) by using the belt 4. Then, the entirety of the forearm B of the patient, the splint unit 1, and the traction unit 20 are covered with the cover 16. The steps of the operation described above are not definite, and the order of the steps may be changed.

As described above, in the first embodiment, the frame 9, the hook-shaped protrusion 11, the knob screw 12, the elastic body 13, the wire or string 14, and the pulley 15 constitute the traction unit 20 for pulling the brace 10. The traction unit 20 is supported by the splint unit 1 through the frame supporting unit 7. The splint unit 1 is fixed or attached to the forearm B of the patient by using the belts 5a and 5b. The splint unit 1 is also attached to the upper arm C of the patient by using the upper arm attachment unit 6 and the belt 6a. Therefore, the traction unit 20 is attached to and supported by the upper limb (the forearm B and the upper arm C) of the patient, so that the fractured part such as the finger E can be continuously pulled while the patient performs daily activities such as walking. In particular, as described above, in the first embodiment, the splint unit 1 and the traction unit 20, which is supported by the splint unit 1, are fixed or attached to the trunk A of the patient by using the belt 4 and the pads 2 and 3. Therefore, traction can be continuously and stably performed irrespective of whether the patient is in an erect position, a seated position, or a recumbent or supine position.

In the first embodiment, the traction force can be easily set and changed by adjusting the degree to which the knob screw 12 is screwed. Therefore, with the first embodiment, the traction force can be applied to a patient in accordance with the position and the state of the fracture and the body size of the patient, and the traction force can be changed in accordance with the progress of the treatment. With the first embodiment, the traction direction can be easily changed during the treatment. Moreover, in the first embodiment, the elastic body 13 including a spring or the like is used. Therefore, with the first embodiment, the traction force is not changed suddenly due to vibration or displacement, so that a negative influence on the treatment due to an excessive force applied to the affected part is prevented.

In the first embodiment, if the patient is only in an erect position or a seated position, the patient can support his/her forearm B, the splint unit 1, and the traction unit 20 only with his/her arm muscles. In this case, it is not necessary for the patient to attach the splint unit 1 and the traction unit 20 to his/her trunk A by using the belt 4 as illustrated in FIG. 1. That is, the patient can hold the splint unit 1 and the traction unit 20 according to the first embodiment only with his/her forearm B, i.e., with his/her arm muscles. However, when using the fracture treatment device for a long time, it is difficult for the patient to support the splint unit 1 and the traction unit 20 only with his/her arm muscles. Therefore, in order that the device can be used easily and stably for a long time, it is preferable that the splint unit 1 and the traction unit 20 be supported by the patient's trunk A or shoulder by using the belt 4 and the like.

In the first embodiment, the splint unit 1, the traction unit 20, and the forearm B of the patient are supported by the trunk A of the patient by attaching the splint unit 1 to the trunk A of the patient by using the belt 4. However, in the present invention, instead of the belt 4, a string or a cloth (a triangular sling or the like) that is slung over the shoulder may be used so that the splint unit 1, the traction unit 20, and the forearm of the patient can be supported by the shoulder of the patient. That is, in the present invention, instead of the belt 4, a known arm holder or an arm strap may be used as an orthopedic appliance that is attached to the shoulder of the patient and that supports the splint unit 1, the traction unit 20, and the forearm B of the patient. Alternatively, in the first embodiment, a known “Shoulder Brace” (trademark, provided by Alcare Co., Ltd. (1-2-1 Kinshi, Sumida-ku, Tokyo, Japan)) may be used as an orthopedic appliance that is attached to the shoulder and the trunk A of the patient so as to support the splint unit 1, the traction unit 20, and the forearm B of the patient. As a further alternative, in the first embodiment, an end of the splint unit 1 may be sewed onto clothes worn by the patient, so that the splint unit 1 is supported at a position in front of the chest or the abdomen of the patient.

In the first embodiment, the traction force is generated by winding a screw (winch mechanism) attached to an end of the wire or string 14. The generated traction force is received by the upper arm C through the upper arm attachment unit 6, which is located slightly above the elbow (i.e., a reaction force of the traction force is generated in the upper arm C). In the present invention, the traction force may be received by the forearm B (for example, a part of the forearm B near to the belts 5a and 5b) instead of the upper arm C. In the first embodiment, the elastic body 13, such as a spring or a rubber, is disposed between the end of the wire or string 14 and the knob screw 12, so that a sudden change in the traction force is prevented.

In the present invention, the wire or string 14 itself may be made of an elastic material to prevent a sudden change in the traction force. In the present invention, in order to prevent a sudden change in the traction force, a spring or the like may be incorporated in the frame or the supporting unit of the frame to which the wire or string 14 is attached. The fracture treatment device according to the first embodiment may be slung over the shoulder or may be attached to a vest-like orthopedic appliance that is worn by the patient.

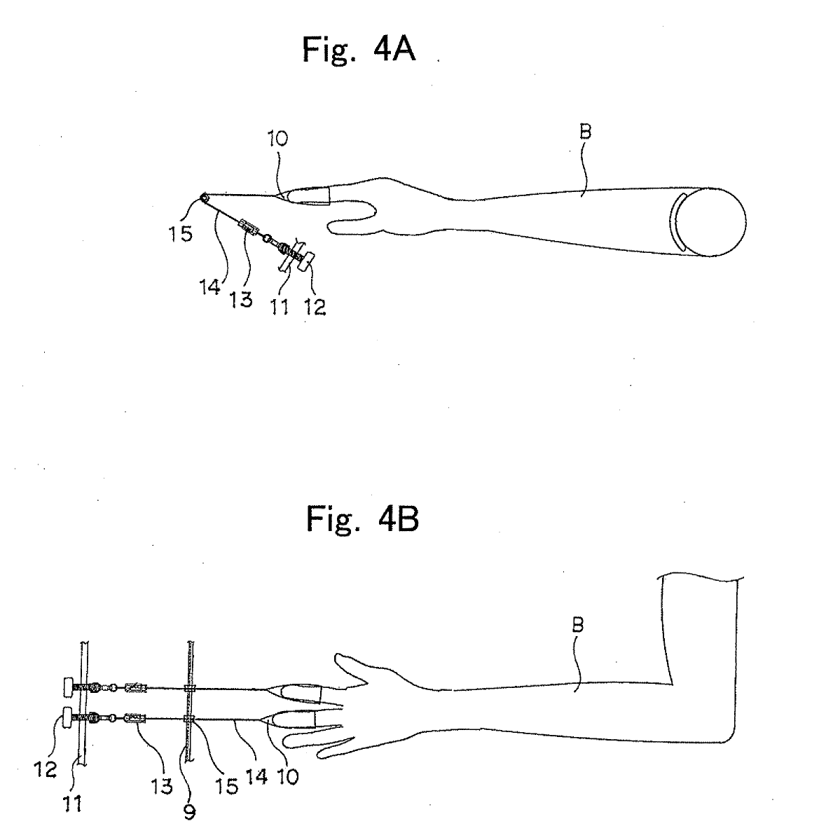

FIGS. 4A and 4B illustrate a first modification of the first embodiment. In the first modification illustrated in FIGS. 4A and 43, in particular as illustrated in FIG. 48, the traction unit 20 includes a plurality of winches (mechanism for adjusting the length of the wire or string 14). In this case, by setting different traction strokes and different traction forces for respective winches, effectiveness of treatment can be increased in particular when there are a plurality of fractured parts.

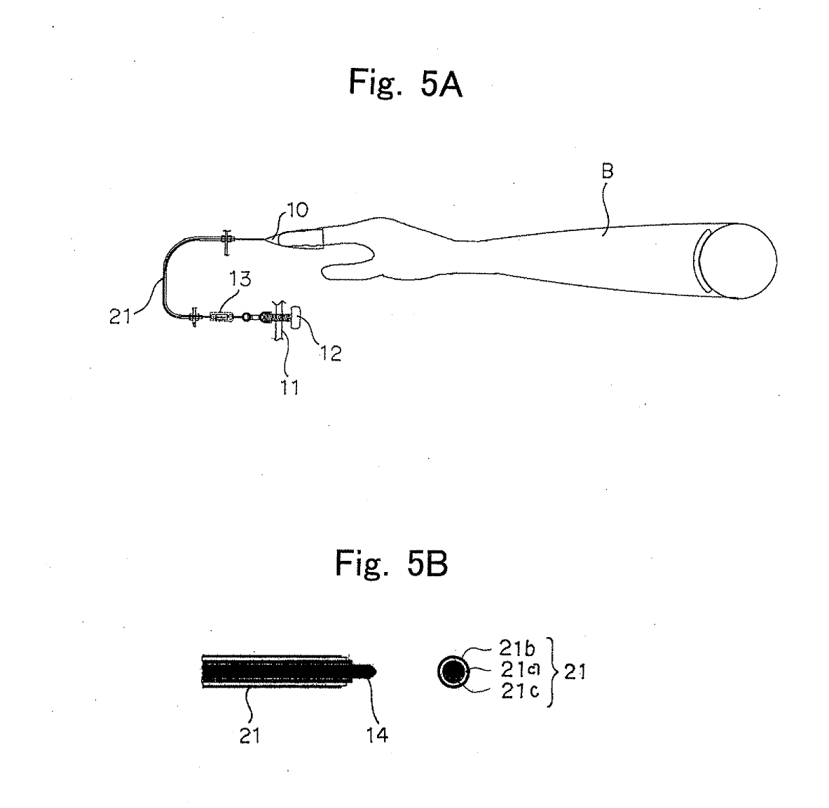

FIGS. 5A and 5B illustrate a second modification of the first embodiment. In the second modification illustrated in FIG. 5A, a guide tube 21 is used as means for changing the direction of the traction force, instead of the pulley 15 illustrated in FIG. 1. The guide tube 21 is substantially U-shaped, and the inner surface of the guide tube 21 is made of a low-friction plastic. The wire or string 14 is inserted through the guide tube 21. The second modification has an advantage in that the safety, the space utility, and the appearance are improved because only a small part of the wire or string 14, which transmits the traction force, is exposed to the outside. FIG. 5B illustrates the structure of the guide tube 21, which includes a tube 21a that is a body of the guide tube 21 and made of a material having a high strength, a covering 21b formed on the outer peripheral surface of the tube 21a, and a low-friction coating 21c that is formed on the inner peripheral surface of the tube 21a.

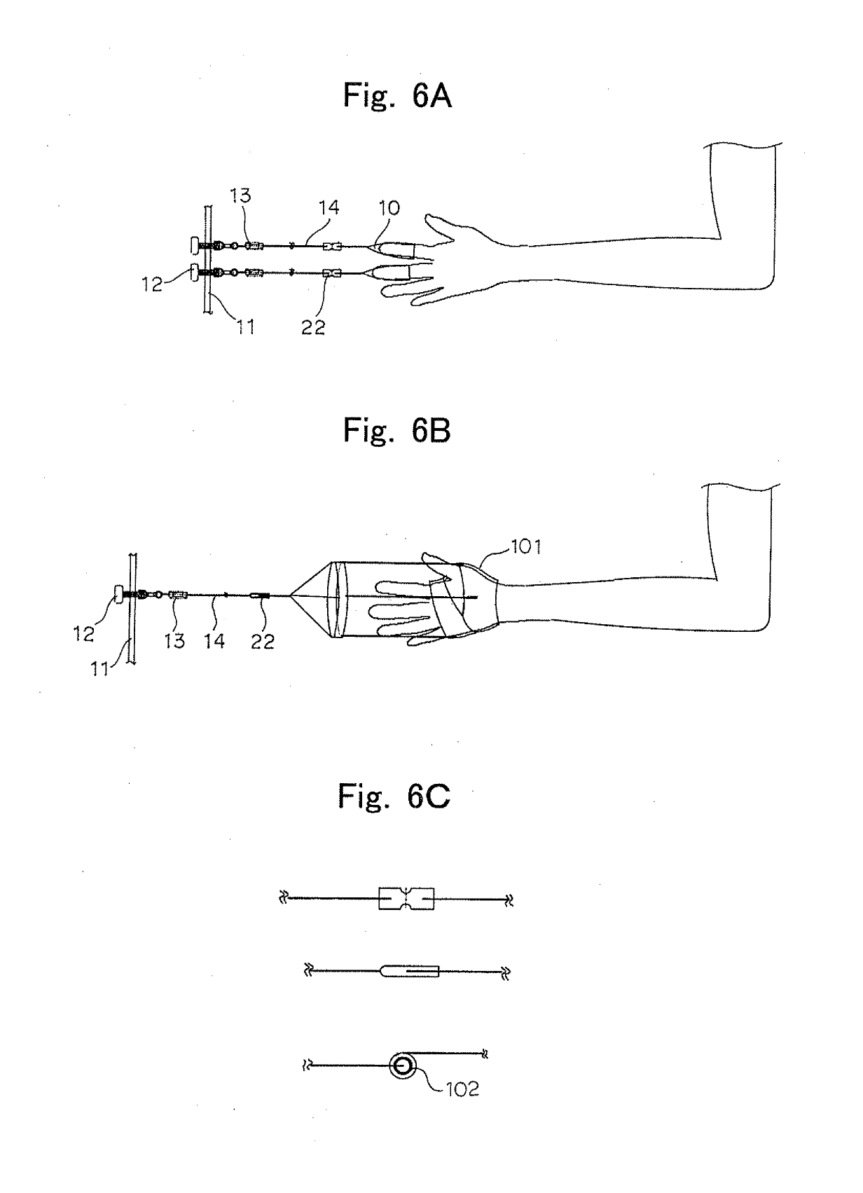

FIGS. 6A to 6C illustrate a third modification of the first embodiment. In the third modification illustrated in FIGS. 6A to 6C, the fracture treatment device includes a clutch mechanism 22 that is disposed in a middle part of the wire or string 14 (between the brace 10 and the elastic body 13 in FIG. 6A). The clutch mechanism 22 serves as a safety device that prevents an excessive traction force from being applied to an affected part. FIG. 6B illustrates an example that includes, instead of the brace 10 (finger trap) for fixing the tip of the finger as illustrated in FIG. 6A, a different type of orthopedic appliance for fixing a wrist and the surrounding part of the patient (for example, a known orthopedic appliance including a polyurethane rubber sheet and a bandage or the like). FIG. 6C illustrates variations of the clutch mechanism 22. The upper one is a breakable clutch (which breaks when a load that is greater than a certain value is applied), the middle one is a friction clutch using frictional resistance (sliding type), and the lower one is a friction clutch (rotary type). FIG. 6C also illustrated a friction surface 102.

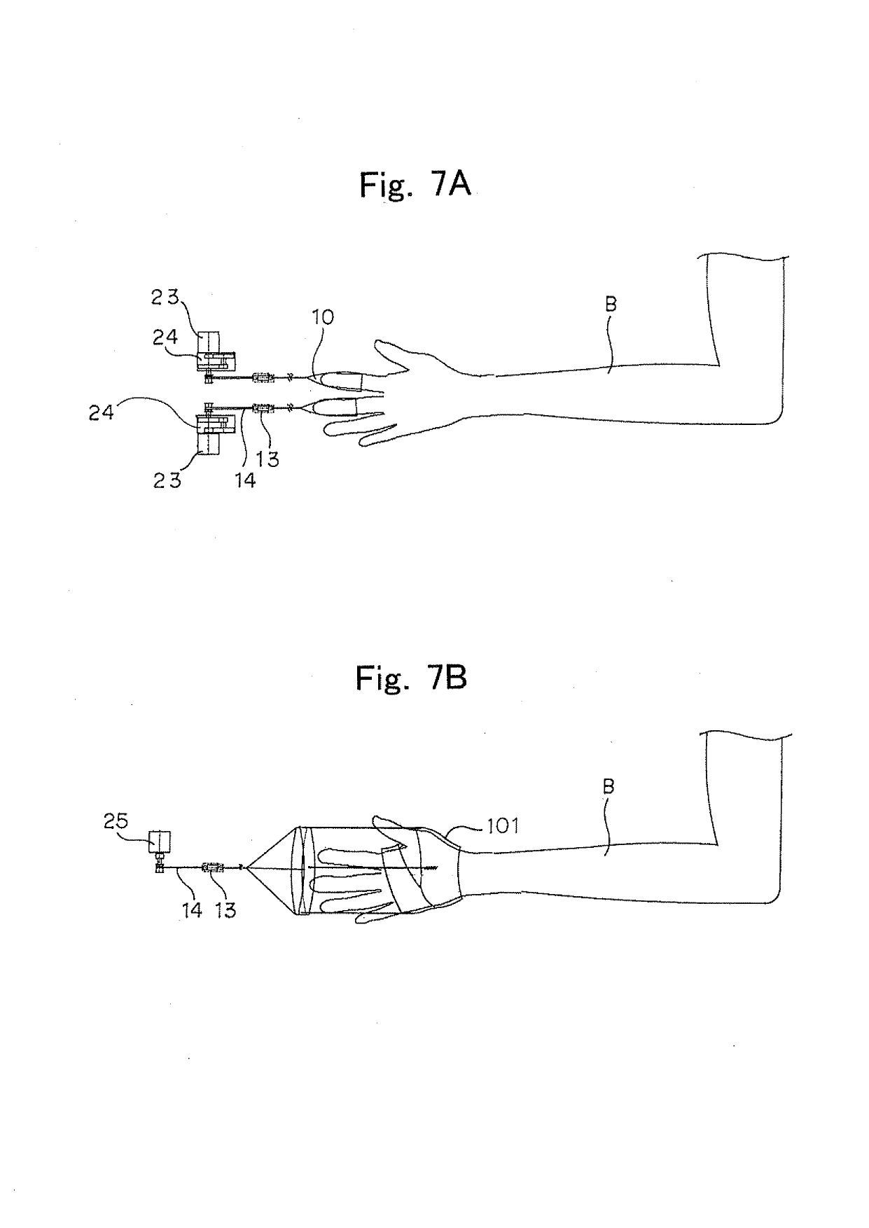

FIGS. 7A and 7B illustrate a fourth modification of the first embodiment. In the fourth modification illustrated in FIGS. 7A and 7B, a motor 23 is used as means for adjusting the length of the wire or string 14 described in the first embodiment, instead of the manually-driven knob screw 12 in the first embodiment. FIG. 7A illustrates an example in which two units, each including the motor 23 and a speed reducer 24, are used (in order to pull two fractured parts). FIG. 7B illustrates an example in which a motor 25 is used to adjust the length of the wire or string 14 in the case where an orthopedic appliance (for example, a known brace including a polyurethane rubber sheet and a bandage or the like), which fixes a wrist and the surrounding part of a patient, is pulled through a stabilizer. Because the motor 25 used in the example of FIG. 7B has high power, the speed reducer 24 used in the example of FIG. 7A is not necessary.

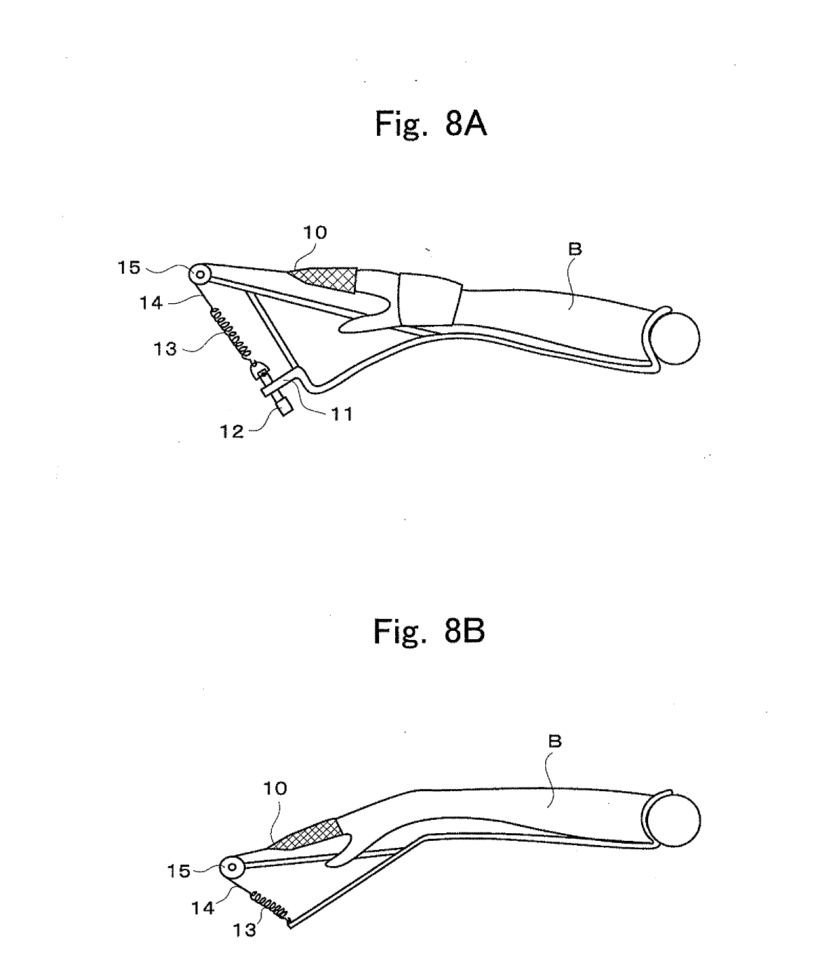

FIGS. 8A and 8B illustrate a fifth modification of the first embodiment. With the fifth modification illustrated in FIGS. 8A and 8B, it is possible to pull a wrist in a state in which the wrist is extended substantially parallel to the longitudinal direction of a forearm B and to pull the wrist in a state in which the wrist is flexed with respect to the longitudinal direction of the forearm B. FIG. 8A illustrates a case where the wrist in pulled in a state in which the wrist is extended parallel to the longitudinal direction of the forearm B, and FIG. 8B illustrates a case where the wrist is pulled in a state in which the wrist is flexed by about 30 degrees with respect to the longitudinal direction of the forearm B. For convenience of drawing, some components are not illustrated in FIG. 8B. With the fifth modification illustrated in FIGS. 8A and 8B, the wrist of the patient can be pulled in a flexed state by changing the position of the pulley 15 from a position at which the wrist of the patient extends parallel to the longitudinal direction of the forearm B to a position at which the wrist forms an angle with respect to the longitudinal direction of the forearm B. The angle is not fixed and may be changed in accordance with the symptom and the treatment policy. Note that a similar structure for changing the position of the pulley 15 from a position at which the wrist of the patient extends parallel to the longitudinal direction of the forearm B to a position on at which the wrist forms an angle with respect to the longitudinal direction has been described with reference to FIG. 1. A wrist brace is shown the fifth modification illustrated in FIGS. 8A and 8B. Traction can be performed by using the wrist brace.

### Second Embodiment

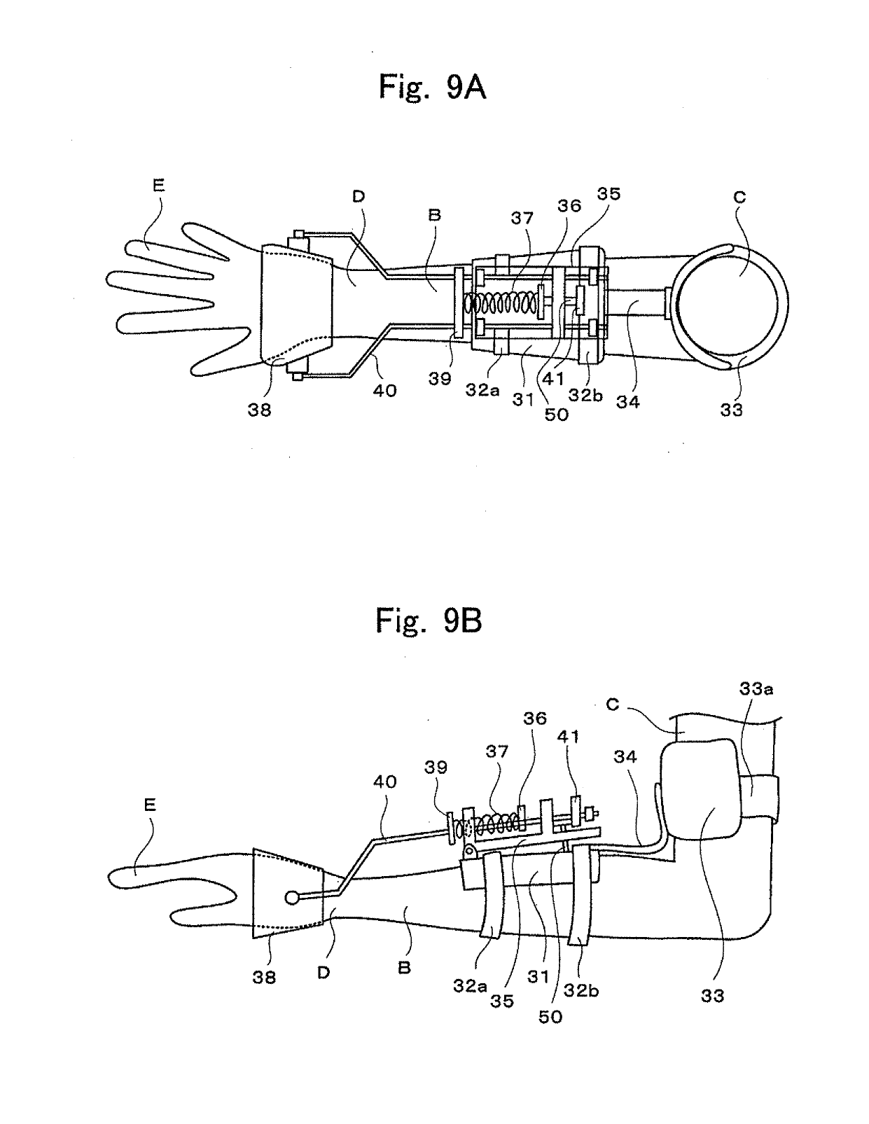

Referring to FIGS. 9A and 9B, a portable or wearable fracture treatment device according to a second embodiment of the present invention will be described. FIG. 9A illustrates the second embodiment attached to a forearm B of a patient as seen from above the forearm B, and FIG. 9B illustrates the second embodiment attached to the forearm B of the patient as seen from a side of the forearm B. In FIGS. 9A and 9B, components the same as those of FIG. 1 are denoted by the same numerals.

FIGS. 9A and 9B illustrate a proximal base unit 31, belts 32a and 32b, an upper arm contact unit 33, a fastener 33a (belt), a coil supporting unit 35, and an upper-arm-side coil fixing unit 36. The proximal base unit 31 has a shape having a substantially semicircular cross-section (a shape that is curved so as to follow the shape of the forearm B of the patient), and is fixed or attached to a part of the patient that is more proximal than the affected part (a part surrounding the forearm B or a substantially central part of the forearm B). The belts 32a and 32b are used to fix the proximal base unit 31 to the forearm B of the patient. The upper arm contact unit 33 is connected to the proximal base unit 31 through a connection unit 34, and is attached to the upper arm C of the patient so as to contact the upper arm C. The fastener 33a (belt) has a substantially ring-like shape that follows the outer peripheral surface of the upper arm C of the patient, and is used to attach the upper arm contact unit 33 and the connection unit 34 to the upper arm C of the patient (so as to prevent the upper arm contact unit 33 and the proximal base unit 31 connected to the upper arm contact unit 33 from being moved toward the wrist D). The coil supporting unit 35 is disposed above the proximal base unit 31 (in a direction away from the forearm B) and is fixed to the proximal base unit 31. The upper-arm-side coil fixing unit 36 is fixed to the coil supporting unit 35 and the proximal base unit 31, and the right end of a coil spring 37 in FIGS. 9A and 9B is fixed to the upper-arm-side coil fixing unit 36. In the second embodiment, the upper arm contact unit 33 serves to transfer a traction force from the traction unit to the upper arm C (so that a reaction force is generated in the upper arm C).

FIGS. 9A and 9B illustrate a distal base unit 38 and a hand-side coil fixing unit 39. The distal base unit 38 has a substantially tapering shape (a cone shape that follows the shape of the back of a hand of the patient), and is fixed to a part of the patient (a wrist or the back of the hand) that is more distal than the affected part. The hand-side coil fixing unit 39 is fixed to the distal base unit 38 through a frame 40, and the left end of the coil spring 37 is fixed to the hand-side coil fixing unit 39.

In the second embodiment, the coil spring 37 is disposed between the upper-arm-side coil fixing unit 36 and the hand-side coil fixing unit 39. The coil spring 37 is a compression spring that generates a force in a direction in which the distance between the upper-arm-side coil fixing unit 36 and the hand-side coil fixing unit 39 is increased (and thereby “a fractured part of the forearm B between the upper-arm-side coil fixing unit 36 and the hand-side coil fixing unit 39” is pulled). FIGS. 9A and 9B illustrate an adjustment screw 41 and an angle adjustment screw 50. The adjustment screw 41 is used to adjust the distance between the upper-arm-side coil fixing unit 36 and the hand-side coil fixing unit 39 (the length of the coil spring 37) so that the force of the coil spring 37 can be adjusted. The angle adjustment screw 50 is used to adjust the angle of the coil supporting unit 35 with respect to the vertical direction of FIG. 9B.

Because the second embodiment has the structure described above, when a part of the forearm B of a patient is fractured, the fractured part can be continuously pulled by attaching the fracture treatment, device according to the second embodiment to the upper limb of the patient, i.e., by fixing or attaching the proximal base unit 31 to a part of the patient between an upper arm and the fractured part and fixing or attaching the distal base unit 38 to a part of the patient between the fractured part and the end of the hand. With the second embodiment, the patient can receive traction treatment very easily, because traction treatment can be performed by only attaching the fracture treatment device to the upper limb of the patient. In the second embodiment, the distal base unit 38 may include a mechanism for adjusting the position at which the frame 40 is attached to the distal base unit 38. In the second embodiment, a length adjustment mechanism such as a turnbuckle may be disposed in a middle part of the frame 40.

The present invention is not limited to the embodiments described above and can be modified in various ways. For example, in the first embodiment, a finger trap is used as the brace 10 for pulling a finger of a patient. However, if a fractured part is in a forearm of the patient, a known orthopedic appliance such as a polyurethane rubber sheet or a bandage may be used to hold or fix the forearm or the like of the patient. In the first embodiment, the splint unit 1 (base unit) can serve as a splint for fixing an affected part of a patient in the case of a fracture. However, an actual splint may be disposed between the splint unit 1 and a forearm B of a patient. In the second embodiment, the coil spring 37 (compression spring) is used as an extension unit that generates a force in a direction in which the distance between the proximal base unit 31 and the distal base unit 38 is increased. However, in the present invention, for example, a motor (linear motor or the like) may be used instead of the coil spring 37 to adjust the force oriented in the direction in which the distance between the proximal base unit 31 and the distal base unit 38 is increased (and the adjusted state may be maintained by using a certain mechanism).

What is claimed is:

1. A portable or wearable fracture treatment device used for treating a forearm fracture, the portable or wearable fracture treatment device comprising:
a splint unit that is attached to an affected part of a forearm of a patient and a part of the patient surrounding the affected part so that the affected part is supported along a longitudinal direction of the forearm;
an upper arm attachment unit that attaches the splint unit to an upper arm of the patient so that the splint unit is supported by the upper arm of the patient;
a fixing unit that fixes a part of the patient that is between the affected part of the forearm and an end of a hand of the patient; and
a traction unit that is supported by the splint unit, the traction unit pulling the affected part of the forearm of the patient in a direction from the affected part of the forearm of the patient toward the hand of the patient by pulling the fixing unit in the direction from the affected part of the forearm of the patient toward the hand of the patient in a state in which the forearm of the patient is flexed substantially horizontally with respect to the upper arm of the patient that extends substantially vertically.

2. The portable or wearable fracture treatment device according to claim 1, further comprising:
a body attachment unit that attaches the splint unit to a trunk or a shoulder of the patient.

3. The portable or wearable fracture treatment device according to claim 2,
wherein the body attachment unit is a belt that is worn around the trunk of the patient, the belt having a front side to which the splint unit is attachable.

4. The portable or wearable fracture treatment device according to claim 2,
wherein the body attachment unit is an orthopedic appliance that is slung over a shoulder of the patient, the appliance having a front side to which the splint unit is attachable.

5. The portable or wearable fracture treatment device according to claim 1,
wherein the traction unit includes a mechanism for adjusting a traction force applied to the affected part, the mechanism adjusting a length of a wire or string whose end is fixed to the fixing unit.

6. The portable or wearable fracture treatment device according to claim 1,
wherein the traction unit includes a motor that generates a force with which the fixing unit is pulled.
